(19) United States
(12) Patent Application Publication
(10) Pub. No.: US 2019/0016027 A1
(43) Pub. Date: Jan. 17, 2019

(54) METHOD FOR PRODUCING AN INJECTION-MOLDED COMPONENT AND CYLINDER FOR A NEEDLE-FREE SYRINGE

(71) Applicant: Gerresheimer Regensburg GmbH, Regensburg (DE)

(72) Inventors: Michael Dehling, Hahnbach (DE); Andreas Knie, Regensburg (DE); Manfred Moritz, Amberg (DE); Christian Lanzl, Bernhardswald (DE)

(21) Appl. No.: 16/029,243

(22) Filed: Jul. 6, 2018

(30) Foreign Application Priority Data
Jul. 13, 2017 (DE) 102017115779.4

(51) Int. Cl. B29C 45/00; B29C 45/26; A61M 5/30

(52) U.S. Cl. CPC B29C 45/0053; B29L 2031/7544; A61M 5/30; B29C 45/261

(57) ABSTRACT

The invention relates to a method for producing an injection-molded part, the injection-molded part being a plastic cylinder for a needle-free syringe. The cylinder has an opening. The invention further relates to an injection-molding tool for producing the cylinder, the injection molded tool including an outer mold component and an inner mold component, which together define a clearance which determine the injection-molded part and the cylinder. The outer mold component and the inner mold component are designed such that the clearance is formed without an obstacle for plastic between the outer mold component and the inner mold component.

### CROSS-REFERENCE TO RELATED APPLICATIONS

The present application is filed under 35 U.S.C. §§ 111(a) and 119 and claims the benefit of German Patent Application No. DE 10 2017 115 779.4, filed on Jul. 13, 2017, which application is incorporated herein by reference in its entirety.

### FIELD

The invention relates to a method for producing an injection-molded component made of plastic, wherein in an opening is formed in the injection-molded component. Furthermore, the invention relates to a needle-free syringe.

### BACKGROUND

In the prior art methods for producing injection-molded parts which form a nozzle or an opening, an inner mold component for the injection molding process is designed to have a projection made of the material of the mold. During the injection molding process, the inner mold component, which consists of the material of the mold, is surrounded with the plastic material, wherein the bore, nozzle or opening of the injection-molded part is formed by means of the projection. In this case, after a few shot numbers (injection events/processes), a breakage of the projection for forming the bore, opening, or nozzle in the injection-molded part occurs. The break, e.g., of the projection in the injection-molding tool, thus does not result in a stable production process, which leads to increased costs, since the downtimes of the machine for the production of injection-molded parts increase.

International patent application WO 2004/069678 A1 discloses a drop dispenser for the controlled dispensing of liquid in the form of drops. The container with the liquid can be provided with an insert dispensing the drops. The drop dispensing insert has a device for restricting the flow rate of the liquid. The outlets for the device for restricting the flow rate of the liquid are drilled with a laser. However, the liquid container itself has no opening that is so small in diameter that it must be drilled with a laser.

European patent EP 2 165 796 B1 discloses a method for forming a micro-hole structure. For this purpose, a working energy source (laser) is projected onto a predetermined drilling site on a surface of a substrate. During the production of the bore, a part of the substrate melts at the bore site with the working energy source. By melting a portion of the substrate, bores are obtained that do not have a uniform diameter over the entire length of the holes.

U.S. Pat. No. 9,125,992 discloses a method for producing a fluid delivery device. The device has a kind of filter barrier which has been formed by means of a laser. By means of the laser, a series of openings are formed in the filter barrier. The openings are thereby formed by means of a laser in the filter barrier in such a way that a series of first cavities is formed, which have a first cross-sectional area and depth which is smaller than the thickness of the filter barrier. Subsequently, a second cavity is formed in each of the first cavities, wherein the at least second cavity has a cross-sectional surface which is smaller than the cross-sectional area of the first cavity. A passage or bore or opening having a constant cross-sectional form along the length of the opening is not provided here.

German Patent Application DE 10 230 451 A1 discloses a method for producing a ventilation opening in a container. The container body is made of a plastic material that absorbs a laser beam having a predetermined wavelength. The ventilation opening is designed as a round bore on the connection piece of the bottle. The container body consists of a plastic material which absorbs the laser beam well and thus can be cut or melted by means of a laser beam.

German patent application DE 10 2012 204 761 A1 discloses the production of soft rubber by means of microwells. The microwells are introduced by laser drilling into the soft rubber profile by means of a laser. The laser can be in particular a pulsed laser.

German Utility Model DE 20 2012 012 535.4 discloses a device for introducing through-holes in a substrate by means of a laser device. The device additionally has elements for collecting the pollutants arising during laser drilling, such as particles, sublimates, aerosols and the like. The collecting device is provided on one side, which is opposite to the side at which the holes are introduced.

European Patent EP 1 752 175 B1 discloses a method for making a needle-free jet injection drug delivery device for needle-free beam injection. The at least one injection nozzle is thereby formed by laser drilling in a corresponding nozzle plate provided for this purpose.

U.S. Pat. No. 4,592,719 mentions, that upon leaving the mold, plastic bottle preforms are engaged internally and/or externally by pneumatic suction grips which form an annular chamber through which atmospheric cooling air is circulated. This increases the rate of production of the preforms by cooling them during handling, rather than separately at a discrete cooling station.

German Patent Application DE 10 2011 077 091 A1 relates to a method for manufacturing curved spectacle frame parts with a defined material thickness distribution. Curved molded sheet parts, in particular from an elastomeric polyurethane plastic, are casted in molds. The distribution of material thickness and curvature of the molded sheet parts being selected in such a manner that each spectacle frame part can be cut out of a respective molded sheet part with the desired curvature and defined material thickness distribution.

### SUMMARY

It is therefore an object of the invention to provide a method for producing an injection-molded parts, with which it is possible to produce injection-molded parts made of plastic and having openings or nozzles with a diameter of <1 mm, wherein the method for producing can be performed in a more robust, durable, and cost-effective manner.

This object is achieved by a method for producing injection-molded parts for needle-free syringes. An injection-molding tool is formed from an outer mold component and an inner mold component, whereby a clearance (free space) is formed between the outer mold component and the inner mold component. A cylinder is formed during the injection process in the clearance, wherein the cylinder contacts, on all sides, an inner surface of the outer mold component and an outer surface of the inner mold component. In the region of the front end of the cylinder, a bore is formed with a beam of a laser.

It is also an object of the invention to provide a cylinder for a needle-free syringe having an opening with a diameter of <1 mm, wherein the production of the cylinder with an injection-molding tool is robust, durable, and inexpensive.

The above object is solved by a cylinder for a needle-free syringe. The cylinder for the needle-free syringe defines a longitudinal axis that is substantially perpendicular to a wall formed at a front end of the cylinder. A bore is introduced into the wall with a laser beam and substantially parallel to the longitudinal axis. The bore has a diameter in the range of 0.015 mm to 1 mm. The bore is designed such that the diameter along the wall remains substantially constant.

According to an embodiment according to the invention, the bore is performed with the laser centrally and substantially parallel to a longitudinal axis in the wall of the cylinder. According to a further embodiment, the laser is chosen in terms of power and/or pulse rate so that the ablated material is sublimated. According to a still further embodiment, the bore introduced into the wall by means of the laser has a diameter in the range of 0.015 mm to 1 mm. The bore produced by means of the laser is designed such that the diameter along a thickness of the wall remains substantially constant.

According to a possible further embodiment of the method, the outer mold component is designed such that a funnel-shaped contour is formed at a front end of the cylinder. By means of the inner mold component, a substantially planar inner surface of the cylinder is formed at a corresponding front end of the inner mold component. The funnel-shaped contour and the planar inner surface define a wall at the front end of the cylinder.

According to an embodiment, again, the bore is performed with the laser centrally and substantially parallel to a longitudinal axis of the cylinder in the wall at the front end of the cylinder. According to an embodiment, the laser is chosen in terms of power and/or pulse rate so that the ablated material is sub-limbed. According to a further embodiment, the bore introduced by means of the laser into the wall has a diameter in the range of 0.015 mm to 1 mm, the bore produced thereby having a substantially constant diameter along the thickness of the wall.

With the injection-molding tool, a cylinder is designed for a needle-free syringe. At a front end of the cylinder, a wall is formed and in the wall, a bore is introduced with a laser beam. The bore substantially has a diameter which has a substantially constant diameter along the thickness of the wall.

According to a possible embodiment, the wall at the front end of the cylinder defines a substantially planar inner surface and a substantially planar outer surface.

According to a possible further embodiment of the wall, a substantially planar inner surface and a funnel-shaped contour are formed at the front end of the cylinder.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a sectional view of an embodiment of an injection-molded part of the prior art, in which at one end the opening or nozzle is formed;

FIG. 2 is a schematic sectional view of an injection-molding tool of the prior art, wherein with the execution of the injection molding process, the opening or nozzle is simultaneously formed in the injection-molded part;

FIG. 3 is a schematic side view of a possible embodiment of the injection-molding tool according to the invention;

FIG. 4 is an exterior view of the injection-molding tool for producing a cylinder for a needle-free syringe;

FIG. 5 is a sectional view of the injection-molded part for the production of the cylinder for needle-free syringes;

FIG. 6 is a sectional view of the cylinder for needle-free syringes, in which the opening or bore is already formed;

FIG. 7 is a side view of a further embodiment of the injection-molding tool for forming a cylinder for needle-free syringes;

FIG. 8 is a sectional view of the further embodiment of the cylinder of the needle-free syringes, formed with the injection-molding tool of FIG. 7; and,

FIG. 9 is an enlarged view of the region A in FIG. 8 of the end of the cylinder for needle-free syringes.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

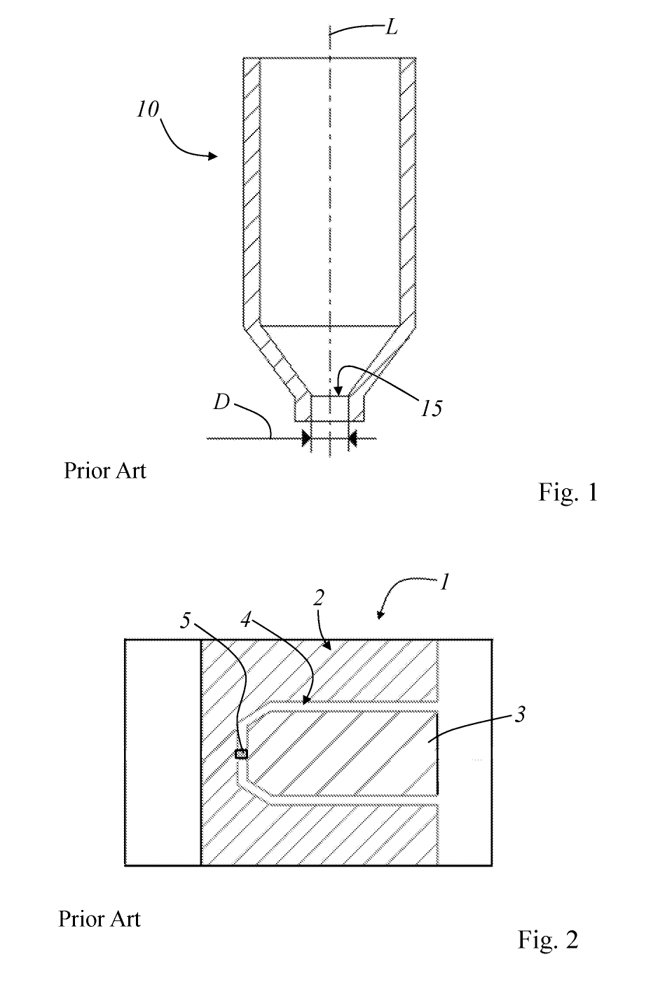

Adverting now to the figures, FIG. 1 shows a schematic sectional view of injection-molded part 10 produced by means of prior art injection-molding tool 1 (see FIG. 2). Due to the corresponding configuration of prior art injection-molding tool 1, opening 15 of injection-molded part 10 is formed. Opening 15 usually has a circular cross-section. Opening 15, or the nozzle, which is produced with injection-molding tool 1 of the prior art, has diameter D which is greater than 1 mm.

FIG. 2 shows a schematic sectional view of injection-molding tool 1 according to the prior art, with which injection-molded part 10 from FIG. 1 can be produced. Injection-molding tool 1 comprises outer mold component 2 and inner mold component 3. When assembling outer mold component 2 and inner mold component 3, clearance 4 is defined between outer mold component 2 and inner mold component 3. To form opening 15 of injection-molded part 10, inner mold component 3 is provided with projection 5. In the assembled state, projection 5 extends between inner mold component 3 and outer mold component 2. When injecting plastic into clearance 4 the space for opening 15 remains free.

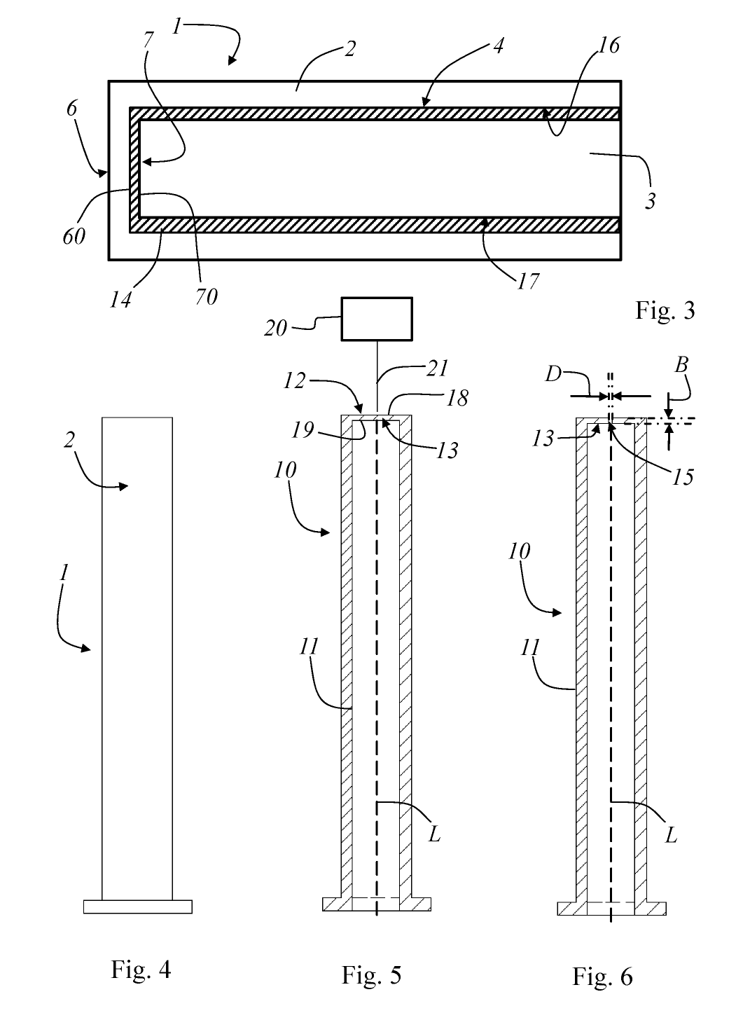

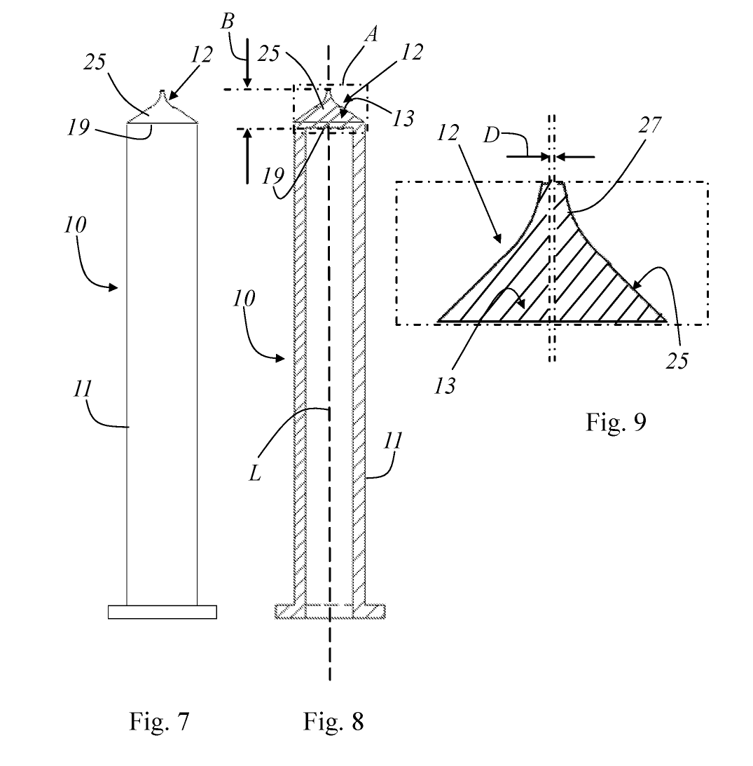

FIG. 3 shows a schematic sectional view of an example embodiment of injection-molding tool 1 according to the invention for producing injection-molded part 10, which is, for example, cylinder 11 for a needle-free syringe. In wall 13 (see FIG. 5 or 6) of cylinder 11 (see FIG. 5 or 6), a corresponding opening 15 is defined. Outer mold component 2 receives inner mold component 3, whereby clearance 4 between inner mold component 3 and outer mold component 2 is formed. Clearance 4 can be filled with plastic 14 during the injection molding process with the plastic material for producing injection-molded part 10 (see FIG. 5). In the example embodiment shown in FIG. 3, a substantially inner planar contour 60 is formed at front end 6 of outer mold component 2. Correspondingly, inner mold component 3 has a substantially outer planar contour 70 formed on a corresponding front end 7 of inner mold component 3. Although in the example embodiment described in FIG. 3, only planar contours 60 and 70 are described, this should not be understood as a limitation of the invention. In particular, it is possible that inner contour 60 of outer mold component 2 can be configured with any desired shape. An example of the possibility of the configuration of contour 60 is illustrated in FIGS. 7 to 9. Clearance 4 of injection-molding tool 1 is substantially defined by inner surface 16 of outer mold component 2 and outer surface 17 of inner mold component 3.

FIG. 4 shows a side view of injection-molding tool 1 for producing an injection-molded part (not shown in FIG. 4). In the illustration shown in FIG. 4, only outer mold component 2 can be seen. In this example embodiment of injection-molding tool 1, injection-molded parts may be produced, e.g. which define the cylinders for needle-free syringes.

FIG. 5 shows a sectional view of injection-molded part 10 which, in the embodiment shown in FIG. 5, may be, for example, a cylinder, i.e., cylinder 11 for a needle-free syringe. In this case, cylinder 11 has already been removed from injection-molding tool 1 (see FIG. 4). Cylinder 11 has a longitudinal axis, i.e., longitudinal axis L which is oriented substantially perpendicular to wall 13 formed at front end 12 of cylinder 11. In the example embodiment of cylinder 11 shown in FIG. 5, wall 13 at front end 12 of cylinder 11 is defined by a substantially planar inner surface 19 and a substantially planar outer surface 18.

Bore 15, illustrated in FIG. 6, is introduced into wall 13 with laser 20 emitting laser beam 21. Laser beam 21 is substantially parallel to longitudinal axis L. Bore 15 introduced with laser 20 in wall 13 has a substantially circular cross-section and has diameter D in the range of 0.015 mm to 1 mm. Bore 15 generated by means of laser 20, which is designed such that along thickness B of wall 13, diameter D does not change substantially.

FIG. 7 shows a side view of another example embodiment of injection-molded part 10, which is, for example, a cylinder, i.e., cylinder 11 for a needle-free syringe. With a special configuration of outer injection-molded component 2, funnel-shaped contour 25 can be formed at the front end of cylinder 11. Inner mold component 3 forms a substantially planar inner surface 19 at a corresponding front end 12 at cylinder 11.

As illustrated in FIG. 8, which is a sectional view of the example embodiment shown in FIG. 7, it can be seen that funnel-shaped contour 25 and planar inner surface 19 define wall 13, at front end 12 of cylinder 11.

FIG. 9 illustrates an enlarged view of the region marked A in FIG. 8. Funnel-shaped contour 25 has formed tapered tip 27 at front end 12 of cylinder 11. In the injection molding process, funnel-shaped contour 25 is formed. Bore 15 of the example embodiment of injection-molded part 10 or of cylinder 11 shown in FIGS. 7-9 is likewise produced with laser 20 in such a way that laser 20 ablates the plastic material by sublimation through wall 13 at front end 12 of cylinder 11. Again, by means of laser 20, bore 15 is formed such that it extends along wall 13 with the substantially constant diameter D.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### LIST OF REFERENCE NUMERALS

1 injection-molding tool
2 outer mold component
3 inner mold component
4 clearance
5 projection
6 front end of outer mold component
7 front end of inner mold component
10 injection-molded part
11 cylinder
12 front end
13 wall
14 plastic
15 bore, hole, opening
16 inner surface
17 outer surface
18 planar outer surface
19 planar inner surface
20 laser
21 laser beam
25 funnel-shaped contour
27 tip
60 inner planar contour
70 outer level contour
A range
B thickness
D diameter
L longitudinal axis

What is claimed is:

1. A method for producing an injection-molded part for a needle-free syringe, the method comprising the steps of:
forming an injection-molding tool from an outer mold component for an injection molding process and an inner mold component for the injection molding process, so that a clearance is formed;
forming a cylinder in the clearance during the injection-molding process, wherein the cylinder contacts, on all sides, an inner surface of the outer mold component and an outer surface of the inner mold component; and,
forming a through-bore in the region of a front end of the cylinder with a beam of a laser.

2. The method of claim 1, wherein a substantially planar outer surface on the cylinder is formed by the outer mold component at a front end thereof and a substantially planar inner surface on the cylinder is formed by the inner mold component at a corresponding front end thereof, the planar outer surface and the planar inner surface defining a wall at the front end of the cylinder.

3. The method of claim 2, wherein the through-bore is formed with the laser centrally and substantially parallel to a longitudinal axis within the wall of the cylinder.

4. The method of claim 3, wherein the laser is selected in terms of power and/or pulse rate so that the ablated material is sublimated.

5. The method of claim 2, wherein the through-bore, introduced in the wall by means of the laser, has a diameter in the range of 0.015 mm to 1 mm, wherein the through-bore, produced by the laser, is designed such that the diameter remains substantially constant along a thickness of the wall.

6. The method of claim 1, wherein a funnel-shaped contour is formed by the outer mold component at a front end of the cylinder and a substantially planar inner surface of the cylinder is formed by the inner mold component at a corresponding front end of the inner mold component, wherein the funnel-shaped contour and the planar inner surface define a wall at the front end of the cylinder.

7. The method of claim 6, wherein the through-bore is performed with the laser centrally and substantially parallel to a longitudinal axis of the cylinder and the wall at the front end of the cylinder.

8. The method of claim 7, wherein the laser is selected in terms of power and/or pulse rate so that the ablated material is sublimated.

9. The method of claim 6, wherein the through-bore, introduced in the wall by the laser, has a diameter in the range of 0.015 mm to 1 mm, wherein the through-bore produced by the laser is designed such that the diameter remains substantially constant along a thickness of the wall.

10. A cylinder for a needle-free syringe, the cylinder comprising:
a wall formed at a front end of the cylinder, the cylinder for the needle-free syringe defining a longitudinal axis substantially aligned perpendicularly to the wall; and,
a through-bore arranged within the wall, the through-bore introduced with a laser beam wherein the through-bore is substantially parallel to the longitudinal axis, and wherein the through-bore has a diameter in the range of 0.015 mm to 1 mm and is designed such that the diameter remains substantially constant along a thickness of the wall.

11. The cylinder of claim 10, wherein the wall at the front end of the cylinder is defined by a substantially planar inner surface and a substantially planar outer surface.

12. The cylinder of claim 10, wherein the wall at the front end of the cylinder is defined by a substantially planar inner surface and a funnel-shaped contour at the front end of the cylinder.
